(19) United States
(12) Patent Application Publication
(10) Pub. No.: US 2017/0212112 A1
(43) Pub. Date: Jul. 27, 2017

(54) INTEGRATION OF SAMPLE SEPARATION WITH RAPID DIAGNOSTIC TESTS FOR IMPROVED SENSITIVITY

(71) Applicant: General Electric Company, Schenectady, NY (US)

(72) Inventors: Bing Li, Clifton Park, NY (US); Matthew Jeremiah Misner, Delanson, NY (US); Gregory Andrew Grossmann, Halfmoon, NY (US); Cathryn Ellen Olsen, Wilton, NY (US); Arunkumar Natarajan, Niskayuna, NY (US)

(21) Appl. No.: 15/005,054

(22) Filed: Jan. 25, 2016

(51) Int. Cl. G01N 33/569

(52) U.S. Cl. CPC G01N 33/56905; G01N 2333/445

(57) ABSTRACT

Provided herein are integrated devices that improve sensitivity of rapid diagnostic tests. The devices described herein integrate sample separation with rapid diagnostic tests and improve signal intensity of the rapid diagnostic tests.

### BACKGROUND

This disclosure relates generally to an improvement in currently available rapid diagnostic tests (RDTs), particularly RDTs for malaria.

A rapid diagnostic test (RDT) is a medical diagnostic test that is quick and easy to perform. RDTs are suitable for preliminary or emergency medical screening, for use in medical facilities with limited resources, and offer a useful alternative to microscopy in situations where reliable microscopic diagnosis is not available or is not available right away. They also allow point-of-care (POC) testing in primary care in situations where formerly only a laboratory test could provide a diagnosis. RDTs do not require clinical diagnostic methods, such as enzyme-linked immunosorbent assay (ELISA) or polymerase chain reaction (PCR), can be performed independent of laboratory equipment by minimally trained personnel, and deliver instant results. RDTs provide results within two hours, and typically provide results in approximately 30 minutes.

A typical RDT employs a dipstick or cassette format. A biological specimen (such as a blood) collected from a patient is applied to a sample pad on the test strip (or card) along with certain reagents. After a length of time (depending on the test), the presence of specific bands in the test strip (card) window indicates whether a certain antigen of interest is present in the patient's sample.

Typically, a drop of sample (e.g., blood) is added to the RDT through one hole (sample well), and then a number of drops of buffer are usually added through another hole (buffer well). The buffer carries the sample along the length of the RDT. In the currently marketed RDTs for malaria, hemoglobin present in red blood cells typically causes background noise on nitrocellulose-based assays for malaria parasites which affects the detection performance (sensitivity) of the test kit. In addition the currently marketed RDTs for malaria utilize only about 5 μL of a blood sample while a finger prick can generate up to 500 μL of sample. The small volume of sample affects detection particularly if the level of analyte in the sample is low and can lead to false negative test results.

U.S. Patent Application Publication No. 20020036170 describes separating plasma from a whole blood sample on a treated membrane, whereby the sample can be assayed on the membrane after separation has occurred. However, the methods described therein comprise the use of material that associates (affinity binding) with the surface of red blood cells and effects separation of the whole blood sample. U.S. Patent Application Publication No. 20130022969 describes lateral chromatography of lysed blood cells to remove hemoglobin. U.S. Pat. No. 5,766,552 discloses the use of an agglutinating agent for clustering of red blood cells. U.S. Pat. No. 8,287,817 discloses separation of blood cells laterally prior to the use of a flow through assay (FTA) by use of hydrophobic carriers affixed to the blood separation zone. In general, the methods disclosed in the art for removal of red blood cells from whole blood employ agents that either lysed cells or induce affinity binding or agglutination of cells. Disadvantages of the methods disclosed in the art include a greater number of steps required for the assay and/or the increased cost of reagents/materials required for the assays. There is a need for improving sensitivity of RDTs, particularly the sensitivity of RDTs for malaria while retaining the lower cost of such tests.

### BRIEF DESCRIPTION

Provided herein are improved rapid diagnostic tests, particularly improved lateral flow assay RDTs. The integrated devices provided herein utilize commercially available or experimental RDTs and improve the RDTs by integrating with the existing RDTs a method for prefiltration of red blood cells from a blood sample by a size-based exclusion and allowing only plasma to reach the sample pads of the RDTs. The removal of red blood cells from the blood sample advantageously allows for the increase of the biological sample volume that reaches the sample pads of the RDTs and reduces interference from hemoglobin of red blood cells, thereby improving the overall signal-to-noise ratio and sensitivity and detection limit of the RDTs as described in more detail below.

In one aspect, provided herein is an integrated device for rapid diagnostic testing of biological samples comprising:
a channeled construct for rapid separation and delivery of a biological sample; and
at least one subsequent lateral-flow strip for performing a binding assay, the lateral-flow strip comprising at least one binding agent deposited thereon with a deposition density that varies periodically along at least a portion of the lateral-flow strip.

Also provided herein are methods for using the integrated device. Further provided herein are channeled constructs and methods of using the channeled constructs.

### DRAWINGS

These and other features, aspects, and advantages of the present invention will become better understood when the following detailed description is read with reference to the accompanying drawings in which like characters represent like parts throughout the drawings, wherein:

FIG. 1 shows modification of a commercially available CTK rapid diagnostic test using the methods described herein.

FIG. 2A and FIG. 2B show modification of a commercially available CareStart rapid diagnostic test using the methods described herein. FIG. 2A shows removal of the pin points. FIG. 2B shows placement of the vertical blood separation membrane.

FIG. 3A and FIG. 3B show modification of a commercially available SD Bioline rapid diagnostic test using the methods described herein. FIG. 3A shows removal of the pin point. FIG. 3B shows placement of the vertical blood separation membrane.

FIG. 4A and FIG. 4B show modification of an experimental rapid diagnostic test that contains cellulose nanobead conjugates (CNBs) using the methods described herein. FIG. 4A shows removal of the ridges. FIG. 4B shows placement of the vertical blood separation membrane.

FIG. 5 shows a comparison of visual intensities for tests run using a modified CTK test and a regular CTK test.

FIG. 6 shows a comparison of visual intensities for tests run using a modified CareStart test and a regular CareStart test.

FIG. 7 shows modification of a commercially available CTK rapid diagnostic test using the methods described herein. (A) placement of L-shaped membrane laterally to the test strip; (B) movement of sample; (C) separation of red blood cell containing front from plasma containing front; (D) plasma reaches sample pad and begin buffer flow.

### DETAILED DESCRIPTION

RDTs are broadly categorized as lateral flow assays (LFAs) or flow through assays (FTAs). A number of currently marketed FTA RDTs are typically designed for detection of multiple pathogens in a single assay (e.g., HIV with Hepatitis B and Hepatitis C). A number of currently marketed RDTs for detection of malarial parasites are LFAs comprising immuno-chromatographic antigen-detection tests, which rely on the capture of dye-labeled antibodies to produce a visible band on a strip of nitro-cellulose. This nitro-cellulose lateral flow test strip is encased in a plastic housing, referred to as cassettes. With malaria RDTs, the dye-labeled antibody first binds to a parasite antigen, and the resultant complex is captured on the strip by a band of bound antibody, forming a visible line in the results window. A control line gives information on the integrity of the antibody-dye conjugate and fluidics of the lateral flow device.

Provided herein are improved RDTs which comprise placing a channeled construct on top of, or lateral to, an existing RDT strip or card. In one embodiment, the cassette housing of the existing RDT is opened and a channeled construct is placed vertically on top of the test window. The cassette housing is then closed. The sample is dropped in the sample window and the vertically placed channeled construct effects an initial separation of the sample by a physical separation, i.e., size exclusion. The channeled construct comprises progressively narrowing channels (elongated pores) which serve to physically filter out red blood cells from a sample while allowing the plasma to flow through and reach the sample pad on the original RDT.

In an alternate embodiment, the cassette housing is opened and an L shaped channeled construct is placed adjacent to the test strip or card such that one edge of the L-shaped channeled construct contacts the lateral flow assay strip or card. The sample is placed on the long end of the L and undergoes a lateral flow chromatographic separation wherein the heavier red blood cells are retained on the channeled construct while the plasma at the leading edge of the sample reaches the sample pad.

While a buffer is typically added to a buffer window in a RDT, such a buffer may also be added after deposition of the sample on the channeled construct.

Typical RDTs are suitable for analysis of about 5 μL of sample, and further, the sample read-out is affected by the presence of hemoglobin from red blood cells. By contrast, the presently described modified RDTs advantageously allow for testing of larger volumes of samples (typically about 75-150 μL of sample), while removing the red blood cells. Thus the analyte read-out is not affected by noise from the presence of hemoglobin. In addition, the ability to process larger sample volume means that a larger volume of analyte reaches the sample pad/test line, thereby improving the signal intensity of the test by up to about ten times the intensity of the test in the absence of the modifications described herein and allowing for detection of very small amounts of analyte which would otherwise be missed in a 5 μL sample. The improved signal intensity is advantageous because RDTs rely on visually detected changes in color on a test strip and a faint color change that is not visually detectable could lead to a false negative on the test.

As used herein, “rapid diagnostic testing” means any testing which can be carried out at the point of care to obtain a quick diagnosis. A rapid diagnostic test (RDT) is a medical diagnostic test that is quick and easy to perform and can be carried out even in the absence of laboratory techniques such as microscopy, ELISA or PCR. RDTs provide results within two hours, typically in approximately 30 minutes. By way of a non-limiting example, rapid diagnostic tests (RDTs) for malaria typically require about 30 minutes from sample collection to result. It will be understood that the time required for an RDT depends on variables such as the sample being tested, the amount of sample, the nature of the analyte and the like, and such variability will be understood by one of skill in the art and is contemplated within the scope of embodiments presented herein.

In addition to integration with lateral flow assay RDTs, the methods and techniques described herein are also suitable for integration with flow through assay RDTs and such flow through assay RDTs are also expressly contemplated within the scope of embodiments provided herein.

As used herein, “channeled construct” means a structure (e.g., a membrane, a ceramic, a glass, and the like) having pores which traverse the construct. The channel may be wide at the top and narrow at the bottom of a construct. A channel may not be continuous in a single direction, i.e., a channel is not always a straight line, a channel may have varied shapes such as the letter “L”, the letter “C”, the letter “S” and the like. Two or more channels may join to form a single channel as the channel(s) traverse the construct.

As used herein, an “anti-lysis coating” stabilizes cells in a biological sample and prevents lysis and release of intracellular components of the cells. In one embodiment, an anti-lysis coating comprises a red blood cell stabilizer.

As used herein, an “asymmetric porous membrane” has larger pores on the upstream side of the membrane which act as a prefilter while the absolute rated downstream side, or exclusion zone, acts as an absolute cut off layer. The graded nature of asymmetric membranes results in a sidedness to the membranes e.g., the top (upstream) side or surface with larger pores and the bottom (downstream) side or surface with smaller pores. In some embodiments, an asymmetric membrane comprises channel pores where the channel pore diameter is larger on the top surface of the membrane and the channel pore diameter is smaller at the lower surface of the membrane thereby allowing for a size-based filtration whereby larger particles/cells are retained in/on the membrane, while the smaller particles/cells/analytes flow through the membrane. Asymmetric membranes may comprise a single layer or multiple layers. The pore size ratio of asymmetric membranes may vary depending on the sample being filtered.

As used herein, “vertically disposed relative to the lateral-flow strip” means that the channeled construct is placed in a plane different from the plane of the test strip and above the test strip. As used herein, “laterally disposed relative to the lateral-flow strip” means that the channeled construct is placed in substantially the same plane as the plane of the test strip.

Provided herein is an integrated device for rapid diagnostic testing of biological samples comprising:
a channeled construct for rapid separation and delivery of a biological sample; and
at least one subsequent test strip for performing a binding assay, the test strip comprising at least one binding agent deposited thereon with a deposition density that varies periodically along at least a portion of the test strip.

In one embodiment, the subsequent test strip is a lateral-flow assay strip. In another embodiment, the subsequent test strip is a flow through assay strip. As used herein, “strip” or “test strip” also includes kits or devices where the “strip” has different dimensions and may be referred to as a “card”.

In one aspect, provided herein is an integrated device for rapid diagnostic testing of biological samples comprising:
a channeled construct for rapid separation and delivery of a biological sample; and
at least one subsequent lateral-flow strip for performing a binding assay, the lateral-flow strip comprising at least one binding agent deposited thereon with a deposition density that varies periodically along at least a portion of the lateral-flow strip.

In one group of embodiments, the biological sample is a sample of blood, feces, sweat, saliva, mucous, milk, urine, semen, serum, plasma, sputum, tears, vaginal fluid, or tissue. In a specific embodiment, the biological sample is a blood sample.

In a group of embodiments, the channeled construct provides rapid separation of blood cells from the blood sample and provides delivery of plasma from the blood sample to the at least one subsequent lateral flow strip.

In some embodiments, the volume of the blood sample tested is from about 75 μL to about 150 μL.

In certain embodiments, the channeled construct comprises a polymer, a ceramic, a glass, a metal, or a combination thereof, for rapid separation and delivery of a biological sample. In certain embodiments, the channeled construct comprises a membrane, a ceramic, a glass, a metal, or a combination thereof, for rapid separation and delivery of a biological sample.

In some embodiments, the channeled construct comprises a membrane for rapid separation and delivery of a biological sample. In some of such embodiments, the membrane is a polymeric membrane. In some of such embodiments, the membrane is coated with an anti-lysis coating.

In certain embodiments, the membrane is an asymmetric porous membrane for rapid separation and delivery of a biological sample. In some of such embodiments, the asymmetric porous membrane is disposed vertically relative to the at least one subsequent lateral flow strip.

In one group of embodiments, the asymmetric porous membrane is a polyethersulfone membrane, a polysulfone membrane, a glass fiber, a nylon membrane, a polyester membrane, a polycarbonate membrane, a polypropylene membrane, a polyvinylidene difluoride membrane, a cellulose membrane, a nitrocellulose membrane, a cellulose acetate membrane, a nitrocellulose mixed ester membrane, a polyurethane membrane, a polyphenylene oxide membrane, a poly(tetrafluoroethylene-co-hexafluoropropylene membrane, a cellulose phosphate membrane, a cellulose/silica gel paper, a borosilicate glass membrane, a quartz membrane, or a combination thereof. In a specific embodiment, the asymmetric porous membrane is an asymmetric polysulfone membrane. In another specific embodiment, the asymmetric porous membrane is an asymmetric polyethersulfone membrane.

In some embodiments, the asymmetric porous membrane has a pore size of about 10 microns to about 100 microns on the top surface with a narrowing of the pore channel to a pore size of about 1 micron to about 3 microns on the bottom surface of the membrane wherein the bottom surface contacts the subsequent lateral flow strip. As used herein, microns and μm are used interchangeably.

In a different group of embodiments, the membrane is disposed laterally relative to the at least one subsequent lateral flow strip. In some of such embodiments, the laterally disposed membrane comprises a cellulose membrane, a nitrocellulose membrane, a glass fiber membrane, a quartz membrane, a borosilicate glass membrane, a mixed cellulose ester membrane, a polyvinylidene difluoride membrane, or a combination thereof, disposed laterally relative to the at least one subsequent lateral flow strip.

In some embodiments, the channeled construct is placed vertically or laterally relative to a commercially available or experimental test strip as described herein in the examples section. In such instances, the flow of the biological sample from the channeled construct to the sample pad/test line/control line of the subsequent lateral flow strip may occur from pressure arising after closure of the housing of the test kit, by capillary force, by gravity, in an electric field, or by any combination thereof. Similarly the flow may be initiated by any such method that initiates contact of the sample with the sample pad/test line/control line of the subsequent lateral flow strip including manually applied pressure (e.g., by pressing down on a certain portion of the housing for the test strip).

In one group of embodiments, the subsequent lateral flow strip comprises a test line comprising a binding agent deposited thereon and a detection line comprising a binding agent deposited thereon. In some embodiments, the binding agent is one or more of an antibody, or a labeled antibody. As used herein, “labeled antibody” includes any labeled antibody which is capable of producing a color change. As such, the antibody may be labeled with a dye, a metal particle (e.g., gold), a compound capable of producing chemiluminescence or fluorescence, or attached to a magnetic bead, a cellulose bead, a polymeric bead labeled with a dye and the like.

In one group of embodiments, the rapid separation of the biological sample is a size exclusion separation.

In some embodiments, provided herein is an integrated rapid diagnostic test device comprising a commercially available or an experimental rapid diagnostic test (RDT) and a blood separation membrane placed laterally or vertically relative to the test strip or card in the RDT. Commercially available RDTs include and are not limited to CTK Biotech, CareStar™, and SD Bioline. Experimental RDTs (e.g., tests comprising cellulose nanobead conjugates) have device layouts similar to commercial ones, but may contain alternative conjugate reporters such as cellulose nanobeads (CNB), various shapes of gold nanoparticles including nanospheres, nanorods, nanoshells, magnetic beads, fluorescence tags, or chemiluminescence molecules. All such alternative conjugate reporters are contemplated within the scope of embodiments presented herein.

In certain embodiments, the blood separation membrane is a vertically disposed asymmetric porous membrane which allows for a physical (non-chemical) size-based exclusion of red blood cells thereby allowing plasma to flow through the blood separation membrane onto the sample pad of the RDT. In other words, the blood separation membrane is designed as a simple funnel filter for red blood cells wherein the red blood cells are unable to flow through the membrane due to narrowing channels which have smaller diameters than the red blood cells themselves. Further, the blood separation membrane is coated with red blood cell stabilizers which prevent lysis of the red blood cells and prevent hemoglobin contamination during the detection step. In other embodiments, the blood separation membrane is a laterally disposed membrane which allows for separation of the slower moving red blood cell front from the plasma front. Examples of such laterally disposed membranes include and are not limited to LF1®, MF1®, VF2®, GF/DVA®, Fusion 5®, and the like.

In one aspect, provided herein is an integrated device for rapid diagnostic testing of blood samples comprising:
an asymmetric porous blood separation membrane for rapid separation and delivery of a blood sample; and
at least one subsequent lateral-flow strip for performing a binding assay, the lateral-flow strip comprising at least one binding agent deposited thereon with a deposition density that varies periodically along at least a portion of the lateral-flow strip;
wherein the asymmetric porous blood separation membrane is disposed vertically relative to the lateral flow strip.

In another aspect, provided herein is an integrated device for rapid diagnostic testing of blood samples comprising:
a laterally disposed blood separation membrane for rapid separation and delivery of a blood sample; and
at least one subsequent lateral-flow strip for performing a binding assay, the lateral-flow strip comprising at least one binding agent deposited thereon with a deposition density that varies periodically along at least a portion of the lateral-flow strip;
wherein at least a portion of the laterally disposed blood separation membrane contacts at least a portion of the subsequent lateral flow strip.

Also provided herein is a method for a rapid assay of a biological sample with an integrated device comprising a channeled construct for rapid separation and delivery of a biological sample; and at least one subsequent lateral-flow strip for performing a binding assay, the lateral-flow strip comprising at least one binding agent deposited thereon with a deposition density that varies periodically along at least a portion of the lateral-flow strip, wherein the subsequent lateral flow strip comprises a sample pad comprising a binding agent deposited thereon and a detection line comprising a binding agent deposited thereon, the method comprising
placing about 75-150 μL of a biological sample on the channeled construct of the integrated device;
allowing the biological sample to traverse the channeled construct to effect a separation of the biological sample; and
allowing the separated analyte-containing plasma to contact the subsequent lateral flow strip.

In one embodiment, the method further comprises adding a buffer to at least a portion of the subsequent lateral flow strip. In another embodiment, the method further comprises allowing the analyte-containing plasma to traverse the subsequent lateral flow strip and recording the result shown on the detection line of the subsequent lateral flow strip. Certain features of the integrated device have been described in detail in preceding paragraphs and it will be understood that all said features of the integrated device are also contemplated for the methods described herein.

Provided herein is a channeled construct for separation of biological samples comprising a porous membrane, wherein said membrane is an asymmetric porous membrane, a membrane comprising affinity surfaces, a membrane comprising hydrophobic cores, or a membrane comprising charged surfaces. In a specific embodiment, the channeled construct is an asymmetric porous membrane. In some of such embodiments, the asymmetric porous membrane is an asymmetric polyethersulfone membrane. In other such embodiments, the asymmetric porous membrane is an asymmetric polysulfone membrane.

Provided herein is a method for rapid separation of biological samples comprising
placing a biological sample on the channeled construct described above;
allowing the biological sample to traverse the channeled construct; and
separating the analyte-containing plasma.

The integrated devices described herein are applicable to a variety of RDTs including RDTs for detection of viruses, infectious diseases, bacteria, cancers, cardiac problems, animal diseases, sexually transmitted diseases, forensics, and the like. Further, the integrated devices described herein may also be further adapted by including additional components such as colorimetric readers, photothermal readers, fluorescence readers, chemiluminescence readers, magnetic readers and the like. The analyte may be amplified prior to detection. While typical RDTs are immune-chromatographic assays which rely on antibody conjugates, dye labeled antibodies, or sandwich assays for detection, other methods of detection are contemplated within the scope of embodiments described herein including and not limited to colorimetric particles (metal particles, polymeric beads labeled with dyes, etc.), fluorescence, chemiluminescence, magnetic beads and the like. In addition to antibody capture, the analyte may be captured by techniques such as nucleotide/aptamer binding and such variants are contemplated as being within the scope of embodiments presented herein. It will be recognized that there are many types of assays such as competitive and non-competitive assays and such variations are also contemplated as being within the scope of embodiments presented herein. Further, multiple detection strips, and/or strips with multiple detection lines may be employed in the devices and methods described herein.

### Examples

All vertical blood separation membranes used herein were commercially available with the average pore size facing up ranging from 30 μm to 100 μm.

### Example 1: Integration of Vertical Channeled Construct with RDTs

### 1) Ctk

Materials: CTK HRP2 device RDT (R0114C); Vertical blood separation membrane (VBSM) (asymmetric polysulfone).

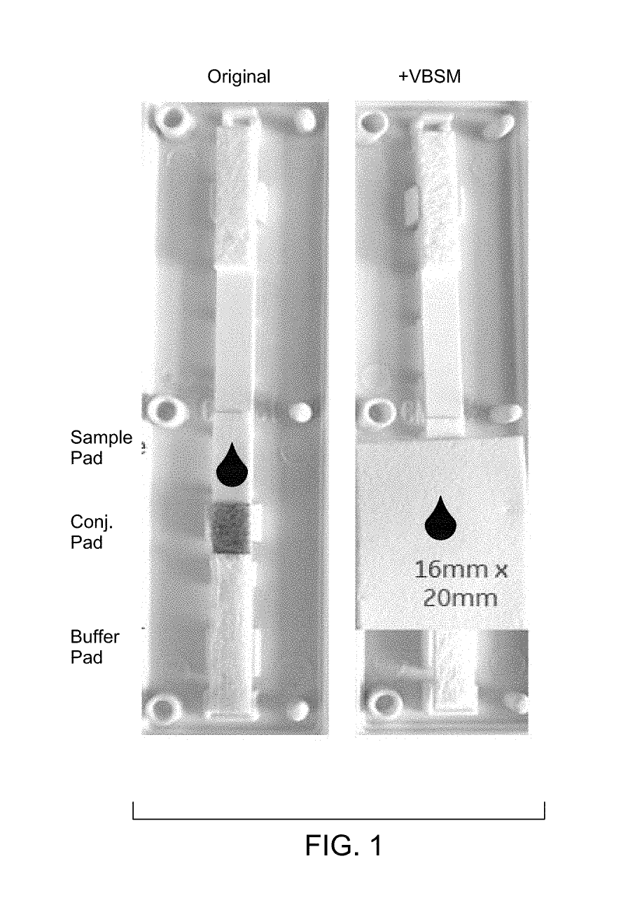

Cut the asymmetric membrane into dimensions of 16 mm×20 mm. Open the CTK housing. Place the membrane on the CTK strip (shiny side facing down), covering sample, conjugate, and part of buffer pads as shown in FIG. 1. Close the CTK housing, or leave it open if being tested shortly.

### 2) CareStart

Materials: CareStart device; Vertical blood separation membrane (VBSM) (asymmetric polysulfone).

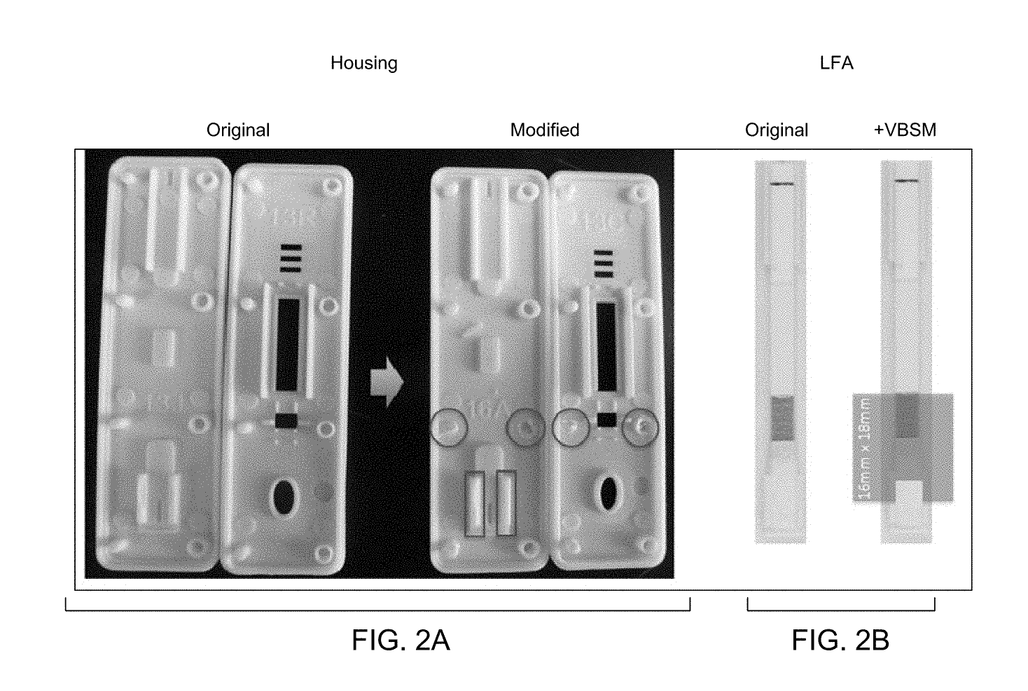

Cut the asymmetric membrane into dimensions of 16 mm×18 mm; Open CareStart housing; Modify housing: remove the second pair of pin point and hole from both the top and bottom plastics, as circled in FIG. 2A; Carefully peel off the buffer pad from conjugate pad, without completely detaching the backing; cut off 5 mm from the upper position. Place VBSM on top of LFA (shiny side facing down), covering conjugate pad, and lay the buffer pad off onto VBSM (FIG. 2B); Close the housing.

### 3) SDBioline

Materials: SDBioline device; Vertical blood separation membrane (VBSM) (asymmetric polysulfone).

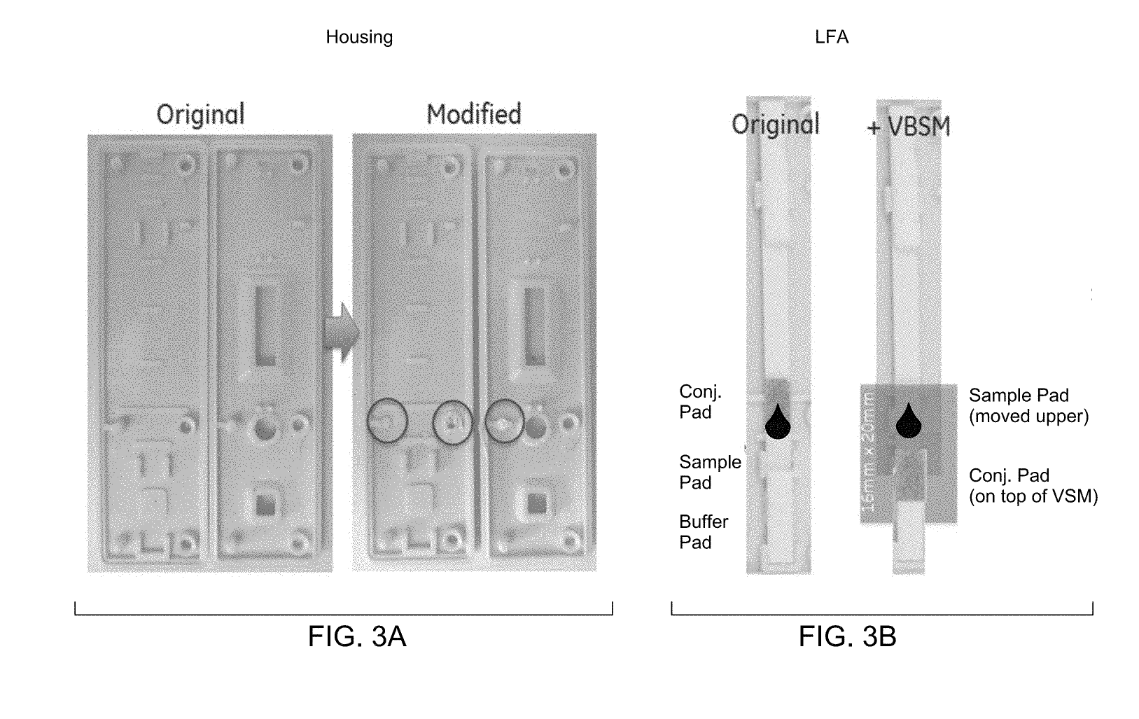

Cut the asymmetric membrane into dimensions of 16 mm×20 mm; Open SD Bioline housing. Modify housing: remove the middle pin point and hole pair from the bottom piece, and the middle pin point form the top cover, as circled in FIG. 3A. Carefully take out conjugate pad and buffer pad; Move sample pad downstream to overlap 1˜2 mm on nitrocellulose (FIG. 3B). Place the membrane on top of the sample pad (shiny side facing down); Place conjugate pad on top of the membrane; Place buffer pad in place, overlapping 1˜2 mm on conjugate pad; Close the housing.

### 4) Cellulose Nanobead Device (CNB)

Materials: an experimental LFA containing cellulose nanobead conjugates; Vertical blood separation membrane (VBSM) (asymmetric polysulfone).

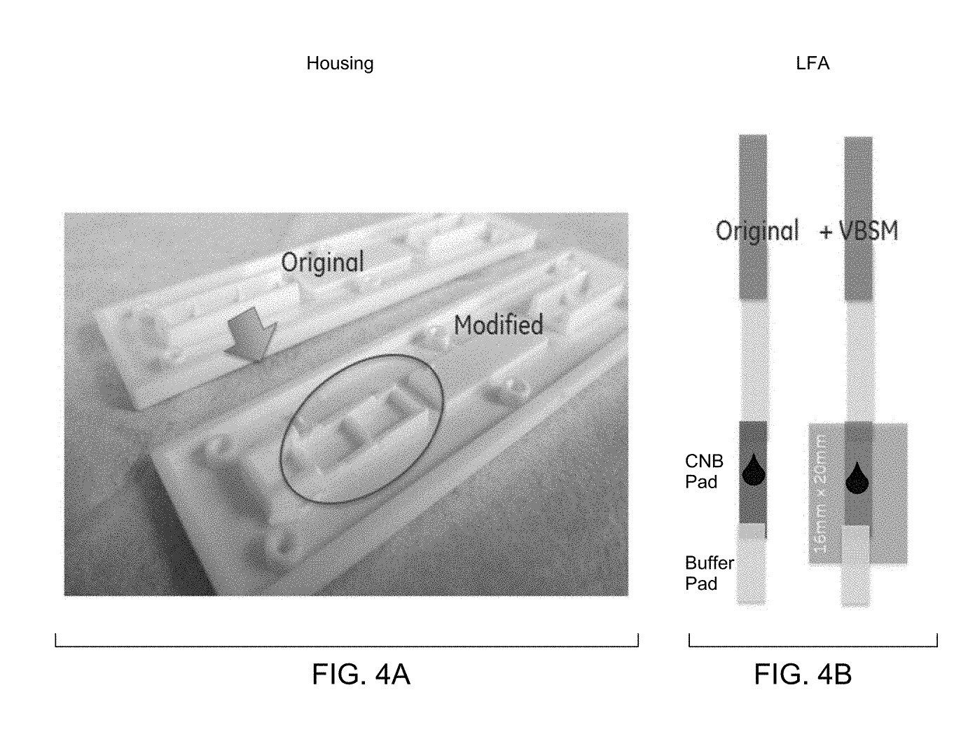

Cut the asymmetric membrane into dimensions of 16 mm×20 mm; Open the housing; Modify housing: remove ⅔ of the ridges from the upstream port of bottom plastic, as indicated in FIG. 4A; Carefully peel off the buffer pad from conjugate pad, without completely detaching the backing; Place the membrane on top of the conjugate pad (shiny side facing down, FIG. 4B); place buffer pad in place. Close the housing.

### Test Procedure:

HRP2 (malaria histidine rich protein 2) stock aliquots were stored under −80° C. Prepare HRP2 dilutions in CPD (citrate phosphate dextrose solution) human whole blood at desired concentrations, and store on ice.

For modified RDTs: Add 75 μL blood sample onto the VBSM through sample port. Chase with 100 μL non-lytic buffer through buffer port. Cover RDT to minimize evaporation from nitrocellulose. Wait and read results after 30 minutes.

For regular RDTs as control: Add 5 μL blood sample through sample port. Chase with 100 μL lateral flow buffer that came with the RDT. Cover RDT to minimize evaporation from nitrocellulose. Wait and read results after 30 minutes.

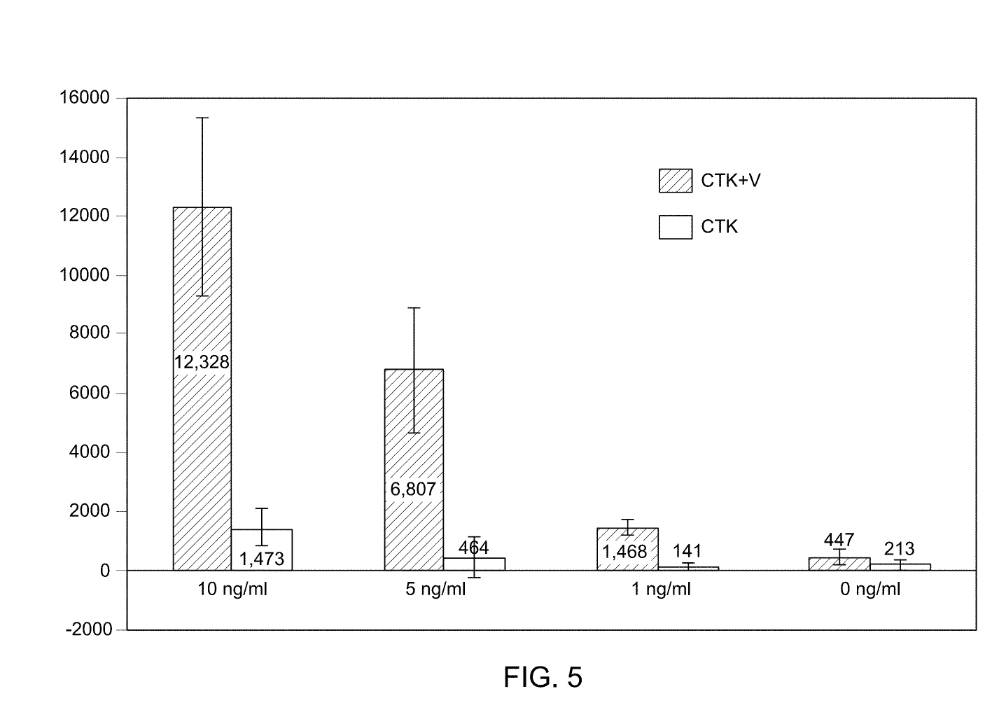

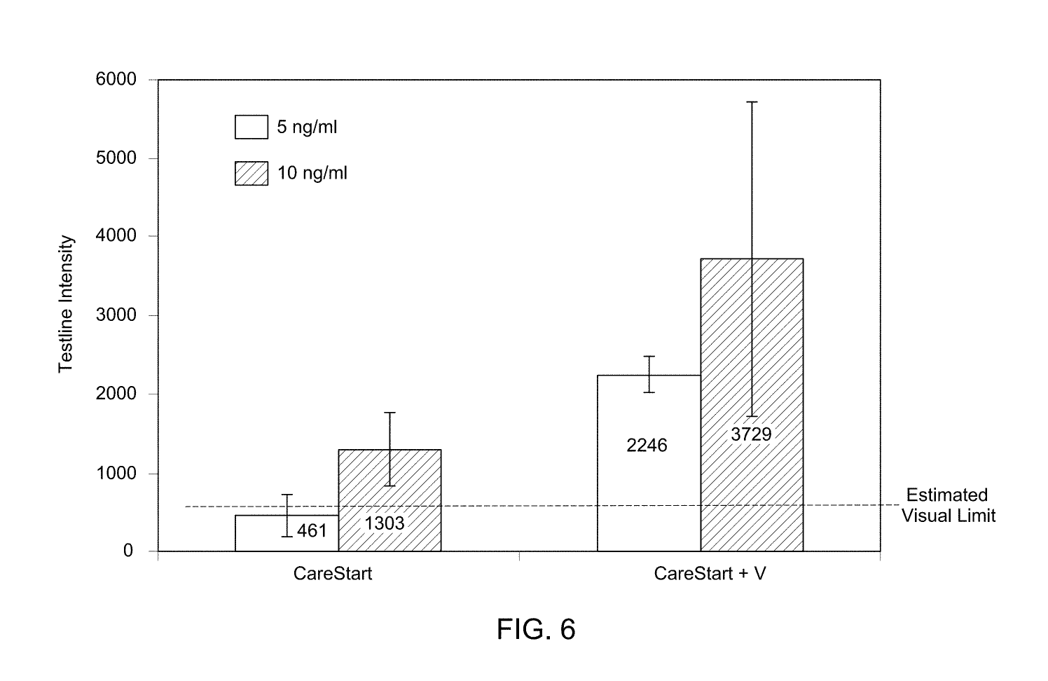

For comparison of modified RDT and regular RDT: Scan images with a flatbed scanner, quantify test line intensities with Image J analysis. Alternatively, results may be read on a photothermal reader. The results are shown in FIG. 5 and FIG. 6.

### Example 2: Integration of Lateral Channeled Construct with RDTs

### 1) CTK/SD Bioline

Materials: CTK/SD Bioline device; lateral blood separation membrane (LBSM) (LF1 from GE).

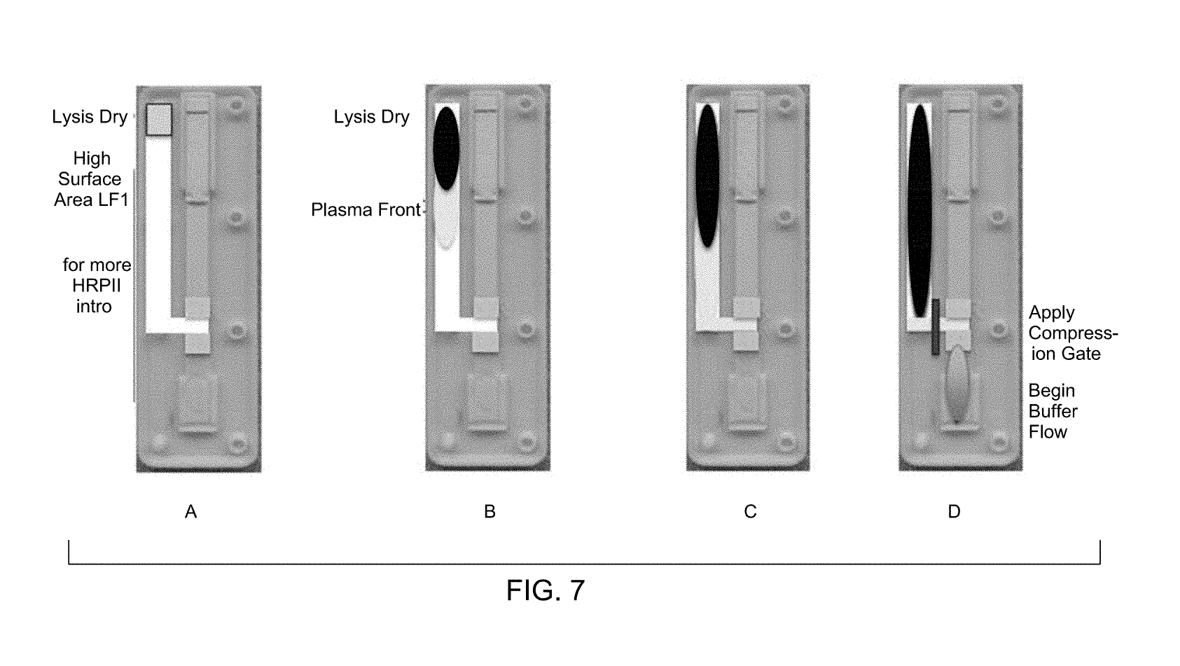

Cut the membrane into dimensions of 40 mm×10 mm (L-shape); Open the housing; Place LBSM on the side of the LFA test strip and the leg (10 mm) of the LF1 material touching the middle of conjugate pad, and part of buffer pads (FIG. 7); Close the housing.

### Test Procedure

HRP2 stock aliquots was stored under −80° C.; Prepare HRP2 dilutions in CPD human whole blood at desired concentrations, and store on ice;

For modified RDTs: Add 75 ul blood sample onto the LBSM at the far end. Allow time for plasma to flow to the conjugate pad and blood to remain on the IBSM. For larger sample volumes, scale LBSM accordingly. Chase with 100 ul non-lytic buffer through buffer port. Cover RDT to minimize evaporation from nitrocellulose. Wait and read results after 20 minutes.

For regular RDTs as control: Add 5 ul blood sample through sample port. Chase with 100 μL lateral flow buffer that came with the RDT. Cover RDT to minimize evaporation from nitrocellulose. Wait and read results after 30 minutes

For comparison of modified RDT and regular RDT: Scan images with a flatbed scanner, quantify test line intensities with Image J analysis. Alternatively, results may be read on a photothermal reader.

### Example 3: Improving Sensitivity of Integrated Devices

Materials: CTK HRP2 device RDT (R0114C); Experimental cellulose nanobeads (CNB) device for HRP2; Vertical blood separation membrane (VBSM) (asymmetric polysulfone).

In this configuration, the original CTK conjugate particle pad is replaced with a cellulose nanobead conjugate pad, and the housing remains the same as original CTK.

Cut VBSM into dimensions of 16 mm×20 mm; Open CTK housing. Take off conjugate pad. Replace CTK conjugate with cellulose nanobeads pad. Place VBSM on top of the modified CTK LFA test strip (shiny side facing down), covering sample, conjugate, and part of buffer pads. Close the CTK housing, or leave it open if being tested shortly.

### Test Procedure

Prepare HRP2 (CTK A3005) dilutions in CPD human whole blood (BioReclamation). Open up the housing, add 75 μL blood sample onto center point of the VBSM slowly. Close the housing, and chase with 100 μL non-lytic buffer through buffer port.

Non lytic buffer 1, for CTK+VBSM: Borate, 0.5% BSA, 0.5% Tween 20, pH 7.4; Non lytic buffer 2, for CNB in CTK+VBSM: PBS, 0.5% BSA, 0.5% Tween 20, pH 9.

Cover RDT to minimize evaporation from detection window. Wait and read results after 30 minutes, optically and/or photothemally. Always run regular RDTs as control by following manufacturer instructions.

It was found that the visual intensity of the CNB conjugate is greater than the visual intensity of the CTK conjugate. Further, about ˜10× improvement in signal intensity was achieved by VBSM.

While only certain features of the invention have been illustrated and described herein, many modifications and changes will occur to those skilled in the art. It is, therefore, to be understood that the appended claims are intended to cover all such modifications and changes as fall within the true spirit of the invention.

1. An integrated device for rapid diagnostic testing of biological samples comprising:
a channeled construct for rapid separation and delivery of a biological sample; and
at least one subsequent lateral-flow strip for performing a binding assay, the lateral-flow strip comprising at least one binding agent deposited thereon with a deposition density that varies periodically along at least a portion of the lateral-flow strip.

2. The integrated device of claim 1, wherein the biological sample is a sample of blood, feces, sweat, saliva, mucous, milk, urine, semen, serum, plasma, sputum, tears, vaginal fluid, or tissue.

3. The integrated device of claim 1, wherein the biological sample is a blood sample.

4. The integrated device of claim 3, wherein the channeled construct provides rapid separation of blood cells from the blood sample and provides delivery of plasma from the blood sample to the at least one subsequent lateral flow strip.

5. The integrated device of claim 3, wherein the volume of the blood sample tested is from about 75 μL to about 150 μL.

6. The integrated device of claim 1, wherein the channeled construct comprises a polymer, a ceramic, a glass, a metal, or a combination thereof, for rapid separation and delivery of a biological sample.

7. The integrated device of claim 1, wherein the channeled construct comprises a membrane for rapid separation and delivery of a biological sample.

8. The integrated device of claim 7, wherein the membrane is coated with an anti-lysis coating.

9. The integrated device of claim 7, wherein the membrane is an asymmetric porous membrane for rapid separation and delivery of a biological sample.

10. The integrated device of claim 9, wherein the asymmetric porous membrane is disposed vertically relative to the at least one subsequent lateral flow strip.

11. The integrated device of claim 9, wherein the asymmetric porous membrane is a polyethersulfone membrane, a polysulfone membrane, a glass fiber, a nylon membrane, a polyester membrane, a polycarbonate membrane, a polypropylene membrane, a polyvinylidene difluoride membrane, a cellulose membrane, a nitrocellulose membrane, a cellulose acetate membrane, a nitrocellulose mixed ester membrane, a polyurethane membrane, a polyphenylene oxide membrane, a poly(tetrafluoroethylene-co-hexafluoropropylene membrane, a cellulose phosphate membrane, a cellulose/silica gel paper, a borosilicate glass membrane, a quartz membrane, or a combination thereof.

12. The integrated device of claim 9, wherein the asymmetric porous membrane is an asymmetric polysulfone membrane.

13. The integrated device of claim 9, wherein the asymmetric porous membrane has a pore size of about 10 microns to about 100 microns on the top surface with a narrowing of the pore channel to a pore size of about 1 micron to about 3 microns on the bottom surface of the membrane wherein the bottom surface contacts the subsequent lateral flow strip.

14. The integrated device of claim 7, wherein the membrane is disposed laterally relative to the at least one subsequent lateral flow strip.

15. The integrated device of claim 14, wherein the membrane comprises a cellulose membrane, a nitrocellulose membrane, a glass fiber membrane, a quartz membrane, a borosilicate glass membrane, a mixed cellulose ester membrane, a polyvinylidene difluoride membrane, or a combination thereof, disposed laterally relative to the at least one subsequent lateral flow strip.

16. The integrated device of claim 1, wherein the subsequent lateral flow strip comprises a test line comprising a binding agent deposited thereon and a detection line comprising a binding agent deposited thereon.

17. The integrated device of claim 1, wherein the binding agent is one or more of an antibody, or a labeled antibody.

18. The integrated device of claim 1, wherein the rapid separation of the biological sample is a size exclusion separation.

19. A method for a rapid assay of a biological sample with an integrated device comprising a channeled construct for rapid separation and delivery of a biological sample; and at least one subsequent lateral-flow strip for performing a binding assay, the lateral-flow strip comprising at least one binding agent deposited thereon with a deposition density that varies periodically along at least a portion of the lateral-flow strip, wherein the subsequent lateral flow strip comprises a sample pad comprising a binding agent deposited thereon and a detection line comprising a binding agent deposited thereon, the method comprising
placing about 75-150 μL of a biological sample on the channeled construct of the integrated device of claim 1;
allowing the biological sample to traverse the channeled construct to effect a separation of the biological sample; and
allowing the separated analyte-containing plasma to contact the subsequent lateral flow strip.

20. The method of claim 19, further comprising adding a buffer to at least a portion of the subsequent lateral flow strip

21. The method of claim 19, further comprising allowing the analyte-containing plasma to traverse the subsequent lateral flow strip and recording the result shown on the detection line of the subsequent lateral flow strip.

22. A channeled construct for separation of biological samples comprising a porous membrane, wherein said membrane is an asymmetric porous membrane, a membrane comprising affinity surfaces, a membrane comprising hydrophobic cores, or a membrane comprising charged surfaces.

23. A method for rapid separation of biological samples comprising
placing a biological sample on the channeled construct of claim 22;
allowing the biological sample to traverse the channeled construct; and
separating the analyte-containing plasma.
